(19) United States
(12) Patent Application Publication
(10) Pub. No.: US 2025/0120743 A1
(43) Pub. Date: Apr. 17, 2025

(54) Autovance Feature of an Intraosseous Device

(71) Applicant: Bard Access Systems, Inc., Salt Lake City, UT (US)

(72) Inventors: Daniel Pett, Sandy, UT (US); Daniel B. Blanchard, Bountiful, UT (US); Eric W. Lindekugel, Salt Lake City, UT (US); Joe Spataro, Cottonwood Heights, UT (US); Ralph Sonderegger, Farmington, UT (US)

(21) Appl. No.: 18/999,553

(22) Filed: Dec. 23, 2024

Related U.S. Application Data
(63) Continuation of application No. 18/075,269, filed on Dec. 5, 2022, now Pat. No. 12,178,471.
Application No. 18/075,269 is a continuation of application No. 17/031,650, filed on Sep. 24, 2020, now Pat. No. 11,517,349.
(60) Provisional application No. 62/907,438, filed on Sep. 27, 2019.

(51) Int. Cl. A61B 17/34; A61B 17/00; A61B 17/16; A61B 90/00

(52) U.S. Cl. CPC A61B 17/3472; A61B 17/1624; A61B 17/3476; A61B 2017/00367; A61B 2090/034

(57) ABSTRACT

An intraosseous system and methods for accessing a medullary cavity. The intraosseous system can include an access assembly and an advancement assembly. The access assembly can include an obturator assembly coupled to a needle assembly. The advancement assembly can include a housing having an inner wall and a drive screw coupled to an energy source. The drive screw can include a threaded portion extending from a drive screw head. The threaded portion can extend into the housing. The advancement assembly can further include an advancement nut threadably engaged with the threaded portion of the drive screw. The advancement nut can include an outer surface configured to slide along the inner wall a predetermined distance without rotating. The advancement assembly can further include a socket configured for attachment to the access assembly.

### PRIORITY

This application is a continuation of U.S. patent application Ser. No. 18/075,269, filed Dec. 5, 2022, now U.S. Pat. No. 12,178,471, which is a continuation of U.S. patent application Ser. No. 17/031,650, filed Sep. 24, 2020, now U.S. Pat. No. 11,517,349, which claims the benefit of priority to U.S. Provisional Application No. 62/907,438, filed Sep. 27, 2019, each of which is incorporated by reference in its entirety into this application.

### SUMMARY

Currently, inserting an intraosseous (“I.O.”) needle often requires a powered drill, or similar powered device that, when activated, drives a needle through a bone layer to access the medullary cavity within. The clinician is required to selectively deactivate the device when they “feel” the needle advance past the relatively hard and compact, cortex layer of the bone and penetrates into the relatively soft, medullary cavity of the bone. However, the relative density of the bone cortex compared with the medullary cavity can vary depending on the bone, size of medullary space, and the patient, e.g. age, health, etc. Accordingly, the clinician relies on a subjective assessment of a lack of resistance in order to determine if the medullary cavity has been successfully accessed. Further, the clinician relies on a subjective assessment to ensure that the needle does not advance through the medullary space and penetrate the far wall of the medullary cavity.

Briefly summarized, embodiments disclosed herein are directed to apparatus and methods for an “autovance” intraosseous device. The device includes a trigger activated system or pressure activated system that causes a needle to advance distally a predetermined distance. The relative thickness of a bone cortex does not vary greatly between patients. Accordingly, the predetermined distance is sufficient to ensure a needle tip extends through the bone cortex, to access the medullary cavity, without penetrating a far wall of the medullary cavity. In some embodiments, the predetermined distance can be between 1 cm and 3 cm, however lesser or greater distances are also contemplated. The advancement can be driven by a spring based system or an electric motor.

Disclosed herein is an autovance intraosseous access device including, a housing, a drive screw, threadably engaged with the housing, the drive screw configured to advance a predetermined distance along a longitudinal axis, and an access assembly coupled to a distal end of the drive screw.

In some embodiments, the autovance intraosseous access device further includes a drive spring or an electric motor configured to rotate the drive screw. In some embodiments, the autovance intraosseous access device further includes a housing nut coupled with the housing in a fixed relationship and configured to threadably engage the drive screw with the housing. In some embodiments, the autovance intraosseous access device further includes a drive spindle rotatably coupled to the housing and slidably engaged with the drive screw to allow the drive screw to advance along the longitudinal axis.

In some embodiments, the autovance intraosseous access device further includes a locking piece coupled to the drive spindle and transitionable between a locked position to inhibit rotation of the drive spindle and an unlocked position to allow the drive spindle to rotate about the longitudinal axis. In some embodiments, a distal surface of the locking piece includes a protrusion, recess, detent, hex-key engagement, star ratchet engagement, or “lock and key” engagement, configured to engage a surface of the housing in the locked position. In some embodiments, the autovance intraosseous access device further includes a drive nut threadably engaged with the drive screw, slidably engaged with the housing and configured to engage the locking piece to transition the locking piece from the locked position to the unlocked position. In some embodiments, the autovance intraosseous access device further includes a drive screw head fixed coupled to a proximal end of the drive screw and configured to engage the housing to inhibit further longitudinal movement of the drive screw. In some embodiments, the predetermined distance is between 1 cm and 3 cm.

Also disclosed is an intraosseous access device including, a housing, a drive screw rotatably engaged with the housing and threadably engaged with an access assembly nut, the drive screw configured to advance the access assembly nut distally by a predetermined distance, and an access assembly coupled to a distal end of the access assembly nut.

In some embodiments, the intraosseous access device further includes a drive spring or an electric motor configured to rotate the drive screw. The drive spring or electric motor is actuated by a trigger actuator where a pressure is applied to the trigger or a pressure actuator where a pressure is applied to a distal tip of the access assembly. The access assembly nut is slidably engaged with the housing to allow longitudinal movement and to inhibit rotational movement of the access assembly nut. In some embodiments, the predetermined distance is between 1 cm and 3 cm.

Also disclosed is an intraosseous access device including, a housing, a drive screw fixedly engaged with the housing and threadably engaged with an access assembly nut, an access assembly coupled to a distal end of the access assembly nut, and an energy source configured to rotate the access assembly nut to advance the access assembly distally a predetermined distance.

In some embodiments, the energy source is a drive spring or an electric motor. The energy source is actuated by a trigger actuator, or a pressure actuator that is actuated when a longitudinal pressure is applied to the access assembly. In some embodiments, the predetermined distance is between 1 cm and 3 cm.

Also disclosed is an intraosseous access device including, a housing, a drive bolt slidably engaged with the housing along a longitudinal axis by a predetermined distance, a biasing member configured to urge the drive bolt distally, an access assembly coupled to a distal end of the drive bolt, and an energy source configured to transition the drive bolt between a locked position and an unlocked position.

In some embodiments, the energy source is a drive spring or an electric motor. The energy source is configured to rotate the drive bolt to transition the drive bolt between a locked position where longitudinal movement of the drive bolt is inhibited, and an unlocked position where the drive bolt can slide longitudinally. In some embodiments, the intraosseous access device further includes a protrusion engaging a surface of a bolt head of the drive bolt in the locked position and the protrusion aligning with a slot extending longitudinally through the bolt head in the unlocked position. The drive bolt engages a portion of the housing or a housing nut to inhibit further distal movement along a longitudinal axis. In some embodiments, the predetermined distance is between 1 cm and 3 cm.

Also disclosed is a method of accessing a medullary cavity including, actuating an energy source, rotating a drive screw, and advancing the drive screw, having an access assembly coupled thereto, a predetermined distance along a longitudinal axis.

In some embodiments, the method further includes actuating a trigger, or applying a longitudinal pressure to the access assembly, to actuate the energy source. In some embodiments, applying a longitudinal pressure to the access assembly includes sliding the drive screw proximally to transition a locking piece from a locked position to an unlocked position, the drive screw including a drive nut threadably engaged therewith and configured to abut against the locking piece. The locking piece engages a surface of a housing in the locked position, the locking piece including one of a protrusion, recess, detent, star ratchet engagement, hex-key engagement, or a “lock and key” engagement to inhibit movement relative to the housing.

In some embodiments, the method further includes rotating a drive spindle coupled to the locking piece, to rotate the drive screw, the locking piece configured to inhibit rotation of the drive spindle in the locked position and allow rotation of the drive spindle in the unlocked position. In some embodiments, a drive screw head, coupled to the drive screw, defines a facet that is slidably engaged with the drive spindle to allow longitudinal movement of the drive screw relative to the drive spindle, and to inhibit rotational movement of the drive screw relative to the drive spindle. The drive screw head engages one of a housing or a housing nut to inhibit further longitudinal movement. The energy source is one of a drive spring or an electric motor. In some embodiments, the predetermined distance is between 1 cm and 3 cm.

Also disclosed is a method of accessing a medullary cavity including, actuating an energy source, rotating a drive screw, and advancing an access assembly nut a predetermined distance along a longitudinal axis, the access assembly nut threadably engaged with the drive screw and including a facet that is slidably engaged with a housing and configured to inhibit rotational movement of the access assembly nut relative to the housing, the access assembly nut including an access assembly coupled to a distal end thereof.

In some embodiments, actuating an energy source includes actuating a trigger or applying a pressure to a distal tip of the access assembly. The energy source is one of a drive spring or an electric motor. In some embodiments, the predetermined distance is between 1 cm and 3 cm.

Also disclosed is a method of accessing a medullary cavity including, actuating an energy source, rotating an access assembly nut including an access assembly coupled to a distal end thereof, the access assembly threadably engaged with a drive screw, and advancing the access assembly a predetermined distance along a longitudinal axis.

In some embodiments, actuating an energy source includes actuating a trigger or applying a pressure to a distal tip of the access assembly. The energy source is one of a drive spring or an electric motor. In some embodiments, the predetermined distance is between 1 cm and 3 cm.

Also disclosed is a method of accessing a medullary cavity including, actuating an energy source, rotating a drive bolt to an unlocked position, and urging the drive bolt distally a predetermined distance, the drive bolt including an access assembly coupled to a distal end thereof.

In some embodiments, actuating an energy source includes actuating a trigger or applying a pressure to a distal tip of the needle. The energy source is one of a drive spring or an electric motor. In some embodiments, the method further includes a biasing member configured to urge the drive bolt distally. Rotating a drive bolt to an unlocked position includes aligning a slot extending longitudinally through a drive bolt head with a protrusion. The drive bolt head engages one of a housing or a housing nut to inhibit further longitudinal movement. In some embodiments, the predetermined distance is between 1 cm and 3 cm.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates an exploded view of an embodiment of an intraosseous access system, wherein an access assembly subset of the system is depicted slightly enlarged and in elevation, and an automated driver component is depicted in perspective, in accordance with embodiments disclosed herein.

FIG. 2 illustrates an intraosseous access system, in accordance with embodiments disclosed herein.

FIG. 3 illustrates an intraosseous access system, in accordance with embodiments disclosed herein.

FIGS. 4A-4B illustrate an intraosseous access system, in accordance with embodiments disclosed herein.

FIG. 5 illustrates an intraosseous access system, in accordance with embodiments disclosed herein.

FIG. 6 illustrates an intraosseous access system, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a needle disclosed herein includes a portion of the needle intended to be near a clinician when the needle is used on a patient. Likewise, a “proximal length” of, for example, the needle includes a length of the needle intended to be near the clinician when the needle is used on the patient. A “proximal end” of, for example, the needle includes an end of the needle intended to be near the clinician when the needle is used on the patient. The proximal portion, the proximal end portion, or the proximal length of the needle can include the proximal end of the needle; however, the proximal portion, the proximal end portion, or the proximal length of the needle need not include the proximal end of the needle. That is, unless context suggests otherwise, the proximal portion, the proximal end portion, or the proximal length of the needle is not a terminal portion or terminal length of the needle.

With respect to “distal,” a “distal portion” or a “distal end portion” of, for example, a needle disclosed herein includes a portion of the needle intended to be near or in a patient when the needle is used on the patient. Likewise, a “distal length” of, for example, the needle includes a length of the needle intended to be near or in the patient when the needle is used on the patient. A “distal end” of, for example, the needle includes an end of the needle intended to be near or in the patient when the needle is used on the patient. The distal portion, the distal end portion, or the distal length of the needle can include the distal end of the needle; however, the distal portion, the distal end portion, or the distal length of the needle need not include the distal end of the needle. That is, unless context suggests otherwise, the distal portion, the distal end portion, or the distal length of the needle is not a terminal portion or terminal length of the needle.

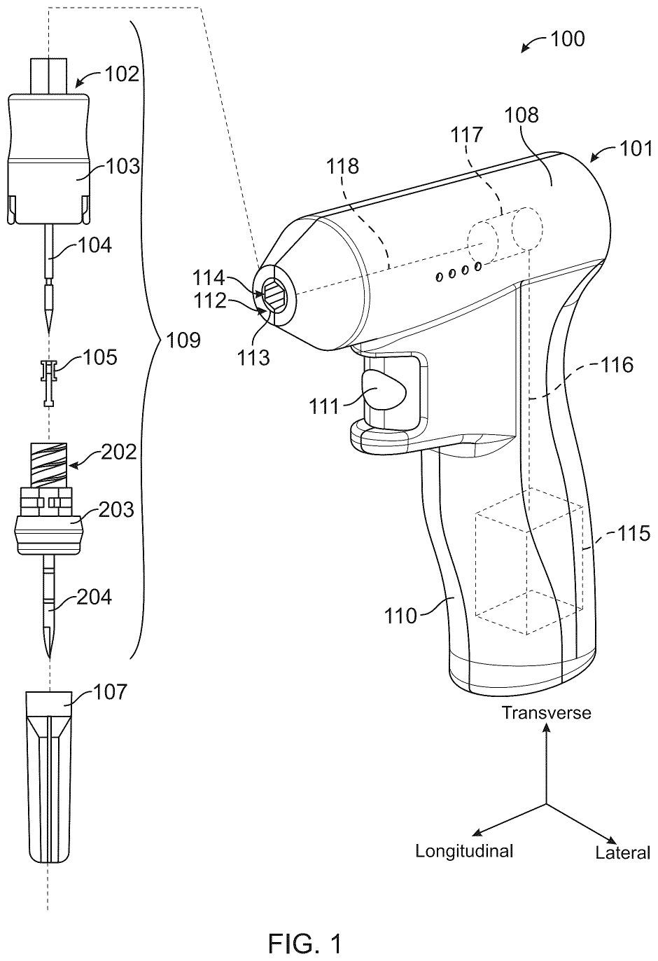

As shown in FIG. 1, and to assist in the description of embodiments described herein, a longitudinal axis extends substantially parallel to an axial length of a needle 204 extending from the driver 101. A lateral axis extends normal to the longitudinal axis, and a transverse axis extends normal to both the longitudinal and lateral axes. As used herein, the term “spring” can include any biasing member including compression springs, torsion springs, flat springs, or the like formed from metal, alloys, plastics, polymers, elastomers, rubber, silicone, composites, combinations thereof, or the like.

Unless defined otherwise, all technical and scientific terms used herein have the same meaning as commonly understood by those of ordinary skill in the art.

The present disclosure relates generally to intraosseous (IO) access devices, systems, and methods thereof. FIG. 1 shows an exploded view of an exemplary embodiment of an intraosseous access system 100, with some components thereof shown in elevation and another shown in perspective. The intraosseous access system 100 can be used to penetrate skin and underlying hard bone for intraosseous access, such as, for example to access the marrow of the bone and/or a vasculature of the patient via a pathway through an interior of the bone.

In an embodiment, the system includes a driver 101 and an access assembly 109. The driver 101 can be used to rotate, or “drill,” the access assembly 109 into a bone of a patient. In embodiments, the driver 101 can be automated or manual. In an embodiment, the driver 101 is an automated driver 108. For example, the automated driver 108 can be a drill that achieves high rotational speeds.

The intraosseous access system 100 can further include an obturator assembly 102, a shield 105, and a needle assembly 202, which may be referred to, collectively, as the access assembly 109. The access assembly 109 may also be referred to as an access system. The obturator assembly 102 is referred to as such herein for convenience. In an embodiment, the obturator assembly 102 includes an obturator 104. However, in some embodiments, the obturator 104 may be replaced with a different elongated medical instrument. As used herein, the term “elongated medical instrument” is a broad term used in its ordinary sense that includes, for example, such devices as needles, cannulas, trocars, obturators, stylets, and the like. Accordingly, the obturator assembly 102 may be referred to more generally as an elongated medical instrument assembly. In like manner, the obturator 104 may be referred to more generally as an elongated medical instrument.

In an embodiment, the obturator assembly 102 includes a coupling hub 103 that is attached to the obturator 104 in any suitable manner (e.g., one or more adhesives or overmolding, etc.). The coupling hub 103 can be configured to interface with the driver 101. The coupling hub 103 may alternatively be referred to as an obturator hub 103 or, more generally, as an elongated instrument hub 103.

In an embodiment, the shield 105 is configured to couple with the obturator 104. The coupling can permit relative longitudinal movement between the obturator 104 and the shield 105, such as sliding, translating, or other movement along an axis of elongation (i.e., axial movement), when the shield 105 is in a first operational mode, and can prevent the same variety of movement when the shield 105 is transitioned to a second operational mode. For example, as further discussed below, the shield 105 may couple with the obturator 104 in a manner that permits longitudinal translation when the obturator 104 maintains the shield 105 in an unlocked state, and when the obturator 104 is moved to a position where it no longer maintains the shield in the unlocked state, the shield 105 may automatically transition to a locked state in which little or no translational movement is permitted between the shield 105 and the obturator 104. Stated otherwise, the shield 105 may be longitudinally locked to a fixed or substantially fixed longitudinal orientation relative to the obturator 104 at which the shield 105 inhibits or prevents inadvertent contact with a distal tip of the obturator, as further discussed below. In various embodiments, the shield 105 may be configured to rotate relative to the obturator 104 about a longitudinal axis of the obturator 104 in one or more of the unlocked or locked states.

With continued reference to FIG. 1, the needle assembly 202 is referred to as such herein for convenience. In an embodiment, the needle assembly 202 includes a needle 204. However, in various other embodiments, the needle 204 may be replaced with a different instrument, such as, for example, a cannula, a tube, or a sheath, and/or may be referred to by a different name, such as one or more of the foregoing examples. Accordingly, the needle assembly 202 may be referred to more generally as a cannula assembly or as a tube assembly. In like manner, the needle 204 may be referred to more generally as a cannula.

In an embodiment, the needle assembly 202 includes a needle hub 203 that is attached to the needle 204 in any suitable manner. The needle hub 203 can be configured to couple with the obturator hub 103 and may thereby be coupled with the driver 101. The needle hub 203 may alternatively be referred to as a cannula hub 203.

In an embodiment, the shield 105 is configured to couple with the needle hub 203. The coupling can prevent relative axial or longitudinal movement between the needle hub 203 and the shield 105, such as sliding, translating, or the like, when the shield 105 is in the first operational mode, and can permit the shield 105 to decouple from the needle hub 203 when the shield 105 is transitioned to the second operational mode. For example, as further discussed below, the shield 105 may couple with the needle hub 203 so as to be maintained at a substantially fixed longitudinal position relative thereto when the obturator 104 maintains the shield 105 in the unlocked state, and when the obturator 104 is moved to a position where it no longer maintains the shield in the unlocked state, the shield 105 may automatically transition to a locked state relative to the obturator 104, in which state the shield 105 also decouples from the needle hub 203.

In an embodiment, the shield 105 can be coupled with the obturator 104, the obturator 104 can be inserted into the needle 204, and the obturator hub 103 can be coupled to the needle hub 203 to assemble the access assembly 109. In an embodiment, a cap 107 may be provided to cover at least a distal portion of the needle 204 and the obturator 104 prior to use of the access assembly 109. For example, in an embodiment, a proximal end of the cap 107 can be coupled to the obturator hub 103.

With continued reference to FIG. 1, the automated driver 108 may take any suitable form. The driver 108 may include a handle 110 that may be gripped by a single hand of a user. The driver 108 may further include an actuator 111, e.g. trigger actuator, of any suitable variety via which a user may selectively actuate the driver 108 to effect rotation of a coupling interface 112. For example, the actuator 111 may comprise a button, as shown, or a switch or other mechanical or electrical element for actuating the driver 108. In an embodiment, the coupling interface 112 is formed as a socket 113 that defines a cavity 114. The coupling interface 112 can be configured to couple with the obturator hub 103. In an embodiment, the socket 113 includes sidewalls that substantially define a hexagonal cavity into which a hexagonal protrusion of the obturator hub 103 can be received. Other suitable connection interfaces are contemplated.

The automated driver 108 can include an energy source 115 of any suitable variety that is configured to energize the rotational movement of the coupling interface 112. For example, in some embodiments, the energy source 115 may comprise one or more batteries that provide electrical power for the automated driver 108. In other embodiments, the energy source 115 can comprise one or more springs (e.g., a coiled spring) or other biasing member that may store potential mechanical energy that may be released upon actuation of the actuator 111.

The energy source 115 may be coupled with the coupling interface 112 in any suitable manner. For example, in an embodiment, the automated driver 108 includes an electrical, mechanical, or electromechanical coupling 116 to a gear assembly 117. In some embodiments, the coupling 116 may include an electrical motor that generates mechanical movement from electrical energy provided by an electrical energy source 115. In other embodiments, the coupling 116 may include a mechanical linkage that mechanically transfers rotational energy from a mechanical (e.g., spring-based) energy source 115 to the gear assembly 117. The automated driver 108 can include a mechanical coupling 118 of any suitable variety to couple the gear assembly 117 with the coupling interface 112. In other embodiments, the gear assembly 117 may be omitted.

In embodiments, the automated driver 108 can rotate the coupling interface 112, and thereby, can rotate the access assembly 109 at rotational speeds significantly greater than can be achieved by manual rotation of the access assembly 109. For example, in various embodiments, the automated driver 108 can rotate the access assembly 109 at speeds of between 200 and 3,000 rotations per minute (rpm). However, it will be appreciated that lesser or greater rotational speeds are also contemplated.

Further details and embodiments of the intraosseous access system 100 can be found in WO 2018/075694, WO 2018/165334, WO 2018/165339, and US 2018/0116693, each of which is incorporated by reference in its entirety into this application.

In an embodiment, the intraosseous access system 100 further includes an advancement assembly disposed between the energy source 115 and the driver coupling interface 112, and operably coupled therewith. When the intraosseous access system 100 is activated, the advancement assembly advances the access assembly 109 a predetermined distance relative to the driver 101, through the longitudinal axis. Since the thickness of the bone cortex is similar between patients, advancing the access assembly 109 the predetermined distance will ensure the tip of the needle 204 advances sufficiently into the medullary cavity to provide access thereto, without impinging a far wall, termed “backwalling.” In an embodiment, the predetermined distance can be between 1 cm and 3 cm, however lesser or greater distances are also contemplated.

In an embodiment, the intraosseous access system 100 is trigger activated, whereby once triggered by a user, the advancement assembly advances the access assembly 109 the predetermined distance before automatically stopping any further advancement. In an embodiment, the intraosseous access system 100 is pressure activated, for example when a pressure is exerted on the needle 204, such as when the needle is pressed against an outer wall of the bone. Once activated, the advancement assembly advances the access assembly 109 the predetermined distance before automatically stopping any further advancement.

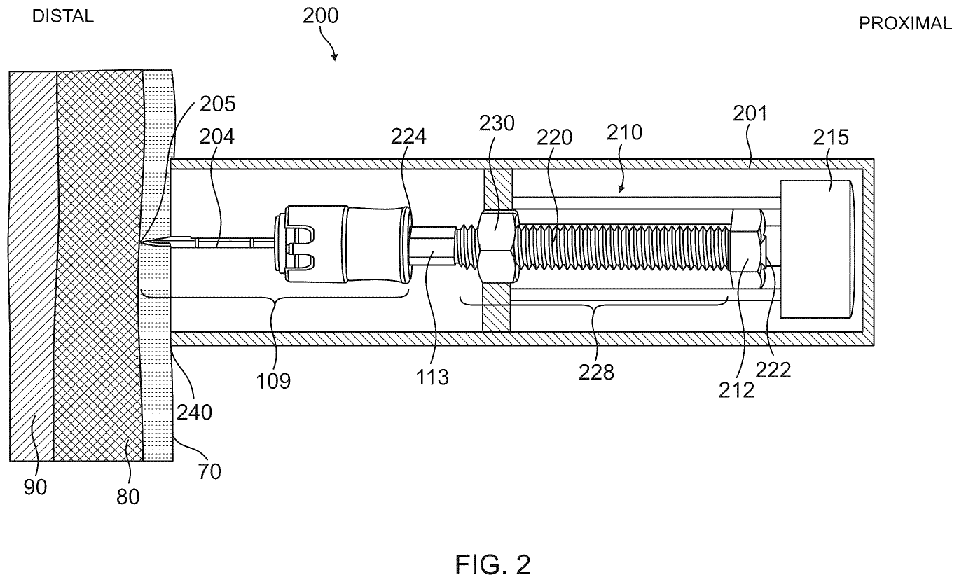

FIG. 2 shows an embodiment of an intraosseous access system 200, including a driver 201, an energy source 215, and an advancement assembly 210. The advancement assembly 210 includes a drive screw 220 extending from a proximal end 222 to a distal end 224 and including a threaded portion 228, and a drive screw head 212 disposed at a proximal end thereof. The drive screw 220 is coupled with an energy source 215 such that the energy source 215 can cause the drive screw 220 to rotate about a longitudinal axis. As described herein the energy source 215 can be an electric motor, battery, torsion spring, combinations thereof, or the like, or similar electrically, chemically, or kinetically powered device that provides sufficient rotational speed and torque. It will be appreciated that the drive screw 220 and energy source 215 can be coupled by way of a gear mechanism, e.g. gear assembly 117.

The drive screw 220 is threadably engaged with a housing nut 230, or similar female threaded structure that engages the male threaded portion 228 of the drive screw 220. The housing nut 230 is fixedly coupled to the driver 201 so as to prevent any relative movement between the housing nut 230 and the driver 201. A distal end 224 of the drive screw 220 is coupled with a socket 113 that engages an access assembly 109. In use, a distal end 240 of the driver 201 is positioned against the skin surface 70 of the patient and the energy source 215 is activated. As discussed herein, the energy source is trigger activated, pressure activated, or combinations thereof. The energy source 215 rotates the drive screw 220, socket 113, and advancement assembly 109 together. The threaded portion 228 of the drive screw 220 engages the housing nut 230 and advances the drive screw 220, socket 113, and advancement assembly 109 in a distal direction relative to the driver 201.

The driver 201 continues to rotate the drive screw 220 until the drive screw head 212 contacts the housing nut 230 and prevents any further distal advancement. In an embodiment, the intraosseous access system 200 includes a switch that shuts off the energy source 215, e.g. an electric motor, when the drive screw head 212 advances a predetermined distance. It will be appreciated that the distance of distal travel of the drive screw 220 and access assembly 109 is sufficient to advance the tip 205 of the needle 204 through the cortex 80 of the bone to access the medullary cavity 90.

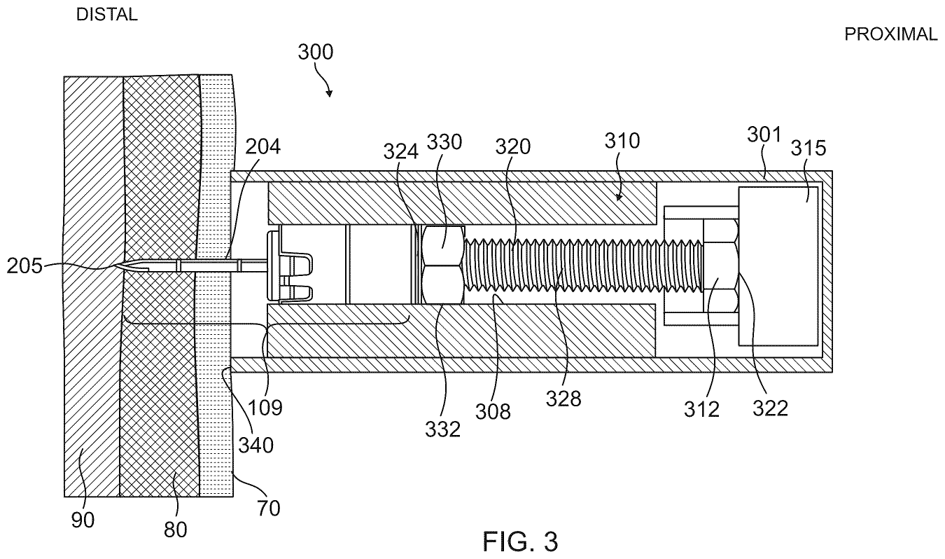

FIG. 3 shows an embodiment of an intraosseous access system 300, including a driver 301, an energy source 315, and an advancement assembly 310. The advancement assembly includes a drive screw 320 extending from a proximal end 322 to a distal end 324. The drive screw 320 includes a threaded portion 328 and a drive screw head 312 disposed at a proximal end thereof. The drive screw 320 is coupled with an energy source 315 such that the energy source 315 can cause the drive screw 320 to rotate about a longitudinal axis. As described herein the energy source 315 can be an electric motor, torsion spring, or the like. The energy source 315 is fixed to a proximal end of the driver 301 such that the drive screw 320 remains stationary relative to the driver 301 along a longitudinal axis.

The drive screw 320 is threadably engaged with an access assembly access assembly nut 330, or similar female threaded structure that engages the male threaded portion 328 of the drive screw 320. The access assembly nut 330 defines one or more side facets 332 that engages an inner wall 308 of the driver 301 such that the access assembly nut 330 is prevented from rotating about a longitudinal axis but is able to slide along a longitudinal axis, relative to the driver 301. An access assembly 109 is coupled to a distal surface of the access assembly nut 330 and optionally includes socket 113, or the like, to secure the access assembly 109 to the access assembly nut 330.

In use, a distal end 340 of the driver 301 is positioned against the skin surface 70 of the patient and the energy source 315 is activated. As discussed herein, the energy source is trigger activated, pressure activated, or combinations thereof. The energy source 315 rotates the drive screw 320 which engages the access assembly nut 330 and drives the access assembly nut 330 and access assembly 109 in a distal direction relative to the driver 301. The energy source 315 continues to rotate the drive screw 320 until the access assembly nut 330 reaches a distal end 324 of the threaded portion 328 and prevents any further distal advancement. In an embodiment, the intraosseous access system 300 includes a switch that shuts off the energy source 315, e.g. an electric motor, when the access assembly nut 330 advances a predetermined distance. It will be appreciated that the distance of distal travel of the access assembly nut 330 and access assembly 109 is sufficient to advance the needle tip 205 through the bone cortex 80 to access the medullary cavity 90, i.e. to provide fluid communication between a lumen of the needle and the medullary cavity 90.

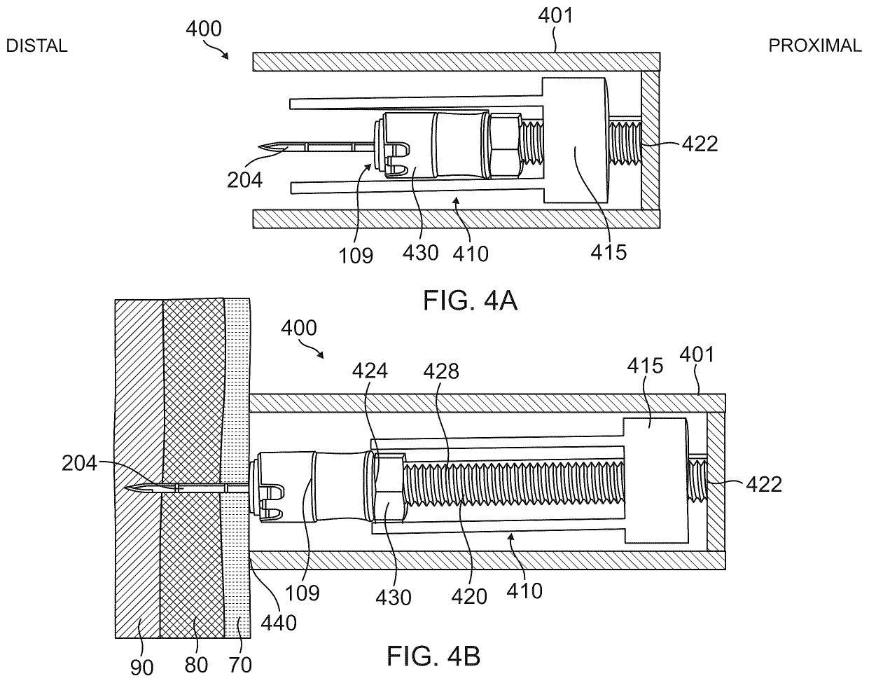

FIGS. 4A-4B shows an embodiment of an intraosseous access system 400, including a driver 401, an energy source 415, and an advancement assembly 410. FIG. 4A shows a retracted position before deployment, FIG. 4B shows a deployed position when the needle has been inserted into the medullary cavity 90. The advancement assembly 410 includes a drive screw 420 extending from a proximal end 422 to a distal end 424 and including a threaded portion 428. The drive screw 420 is attached to the driver 401 so as to prevent any relative movement between the drive screw 420 and the driver 401. An access assembly nut 430 is rotatably coupled with an energy source 415, e.g. electric motor, torsion spring, etc. such that when the energy source 415 is activated, the access assembly nut 430 is rotated relative to the drive screw 420. An access assembly 109 is coupled to a distal surface of the access assembly nut 430 and optionally includes socket 113, or the like, to secure the access assembly 109 to the access assembly nut 430. In an embodiment, the access assembly nut 430 is slidably engaged with the energy source 415 such that the access assembly nut 430 can move along a longitudinal axis relative to the energy source 415. In an embodiment, the energy source 415 is slidably engaged with the driver 401 such that the energy source 415 and access assembly nut 430 can move together along a longitudinal axis relative to the driver 401.

In use, a distal end 440 of the driver 401 is positioned against the skin surface 70 of the patient and the energy source 415 is activated. As discussed herein, the energy source is trigger activated, pressure activated, or combinations thereof. The energy source 415 rotates the access assembly nut 430 and advancement assembly 109 together. The access assembly nut 430 engages the threaded portion 428 of the drive screw 420 and advances the access assembly nut 430 and advancement assembly 109 in a distal direction relative to the driver 401.

The energy source continues to rotate the access assembly nut 430 until the access assembly nut 430 reaches a distal end 424 of the threaded portion 428 and prevents any further distal advancement. In an embodiment, the intraosseous access system 200 includes a switch that shuts off the energy source 415, e.g. an electric motor, when the access assembly nut 430 advances to a predetermined position. It will be appreciated that the distance of distal travel of the access assembly nut 430 and access assembly 109 is sufficient to advance the tip of the needle 204 a predetermined distance, through the cortex 80 of the bone to access the medullary cavity 90.

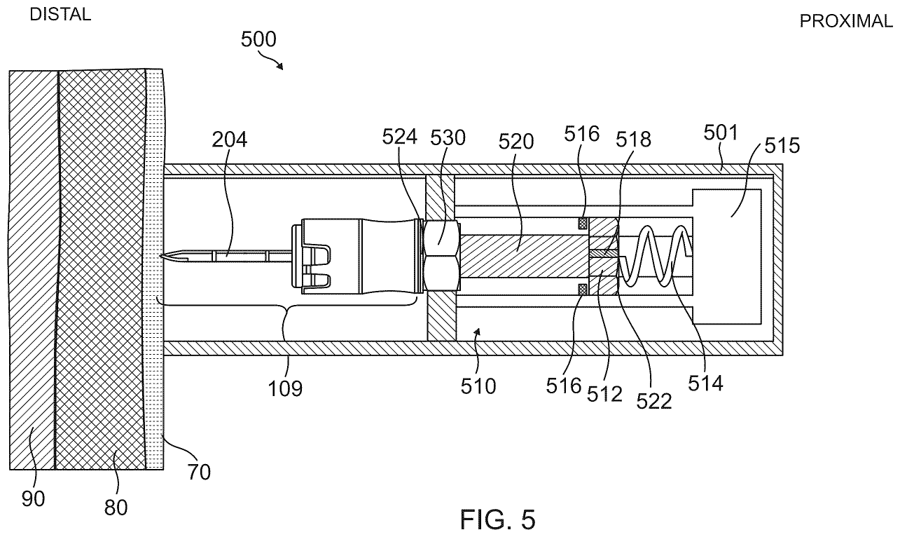

FIG. 5 shows an embodiment of an intraosseous access device 500, including a driver 501, an energy source 515, and an advancement assembly 510. The advancement assembly 510 includes a drive bolt 520, extending from a proximal end 522 to a distal end 524. The drive bolt 520 includes a bolt head 512 disposed at a proximal end 522 and an access assembly 109 coupled to a distal end 524. Optionally, the access assembly 109 is coupled to the drive bolt 520 using socket 113. The drive bolt 520 is slidably engaged with a housing nut 530, or similar annular structure that surrounds the shaft of bolt 520. The housing nut 530 is attached to the driver 501 so as to prevent any relative movement between the housing nut 530 and the driver 501.

A compression spring 514, or similar biasing member, is compressed between a proximal surface of the bolt head 512 and proximal portion of the driver 501, or energy source 515. The spring 514 and bolt head 512 are held in place by a locking mechanism such as one or more protrusions 516. The locking mechanism can transition between a locked position where distal longitudinal movement of the drive bolt 520 is inhibited, and an unlocked position where the drive bolt 520 can slide longitudinally. In a locked position, the protrusions 516 engage a distal surface of the drive bolt head 512 to inhibit distal longitudinal movement. In the unlocked position, the protrusions 516 are aligned with slots 518 extending longitudinally through the drive bolt head 512 and configured to allow the protrusions to slide therethrough. In an embodiment, the drive bolt 520 can be rotated between a locked position and an unlocked position.

In use, the energy source 515 is activated, either by a trigger or pressure activation as described herein. The energy source 515 rotates the bolt head 512 about a longitudinal axis until the protrusions 516 align with the slots 518. This allows the bolt head 512 to slide, relative to the driver 501, along a longitudinal axis. This also allows the compression spring 514 to expand, urging the drive bolt 520 and access assembly 109 in a distal direction for a predetermined distance, until the bolt head 512 abuts against the housing nut 530, preventing any further distal movement. This forces the needle 204 a predetermined distance through the bone cortex 80 until a distal tip thereof extends sufficiently into the medullary cavity 90. It will be appreciated that other locking mechanisms configured to retain the bold head 512 in the retracted position are also contemplated. Exemplary locking mechanisms can include retractable protrusions that slide radially outward perpendicular to the longitudinal axis, pawls, ratchets, catches, grips, combinations thereof, or the like configured to release the bolt head 512 and allow the spring 514, or similar biasing member, to urge the access assembly 109 distally.

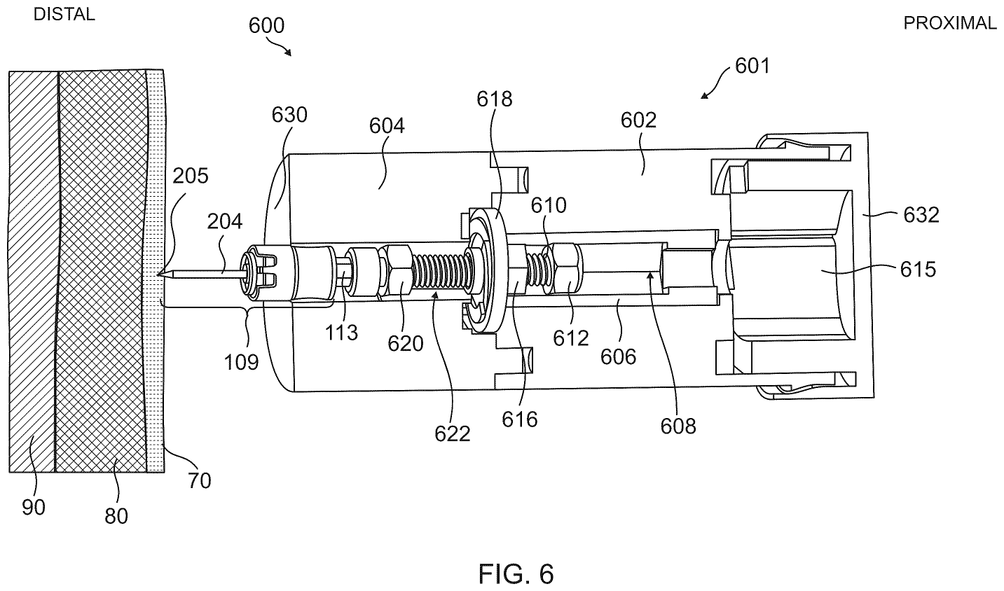

FIG. 6 shows an embodiment of an intraosseous access system 600, which includes a spring driven energy source and a pressure trigger. The intraosseous access system 600 includes an upper housing 602 that engages a lower housing 604 to form a body of driver 601. The driver 601 is substantially radially symmetrical about a longitudinal axis. The upper housing 602 includes an energy source 615, e.g. a drive spring, and a drive spindle 606 that is coupled to the drive spring energy source 615. In an embodiment, the drive spring is a torsion spring, however it will be appreciated that the energy source 615 can also include flat springs, clock springs, electrical motors, and the like, without limitation.

The upper housing 602 and the drive spindle 606 are rotatably engaged such that when the drive spring energy source 615 is activated the drive spindle 606 can rotate freely within the upper housing 602. The drive spindle 606 further includes a centrally disposed, cylindrically shaped cavity 608. A cross-sectional area of the drive spindle cavity 608, extending normal to the longitudinal axis, defines a substantially hexagonal shape that is configured to receive a drive screw head 612. It will be appreciated that the cross-sectional shape of the drive spindle cavity 608 can also include other faceted polygonal cross sectional shapes that are configured to receive the drive screw head 612. In an embodiment, the drive screw head 612 is formed as a single unitary structure with the drive screw 610. In an embodiment, the drive screw head 612 includes a locking nut (not shown) that is threadably engaged with the drive screw 610 and fixed in place so as not to move relative to the drive screw 610. Accordingly, when the drive spring 615 is activated, the drive spindle 606 engages the drive screw head 612 and rotates the drive screw 610. The drive spindle cavity 608 allows the drive screw head 612 to slide relative to the drive spindle 606 along the longitudinal axis.

A distal end of the drive screw 610 engages socket 113, which in turn engages access assembly 109. A housing nut 616 is threadably engaged with the drive screw 610. The housing nut 616 is fixed within the upper housing 602, lower housing 604, or combinations thereof, so as not to move relative to the driver 601. Accordingly, as the drive screw 610 rotates about the longitudinal axis, the threaded engagement with the housing nut 616 causes the drive screw, and access assembly 109, to move longitudinally relative to the driver 601. In an embodiment, the drive screw 610 can advance distally along the longitudinal axis, until the drive screw head 612 engages the housing nut 616, inhibiting further distal movement. In an embodiments, the drive screw can advance a predetermined distance. In an embodiment, the predetermined distance can be between 1 cm and 3 cm, however lesser or greater distances are also contemplated.

The lower housing 604 further includes a centrally disposed, cylindrically shaped cavity 622. A cross-sectional area of the lower housing cavity 622, extending normal to the longitudinal axis, defines a substantially hexagonal shape that is configured to slidably receive a drive nut 620. It will be appreciated that the cross-sectional shape of the lower housing cavity 622 can also include other faceted, polygonal, cross-sectional shapes that are configured to receive the drive nut 620. The drive nut 620 is threadably engaged with the drive screw 610 and disposed adjacent a distal surface of a locking piece 618. Accordingly, when the drive screw 610 rotates, the drive nut 620 is prevented from rotating by the engagement between a facet of the drive nut 620 and a wall surface of the lower housing cavity 622. However, the drive nut 620 is capable of sliding along a longitudinal axis relative to the lower housing 604.

A locking piece 618 is disposed between the drive spindle 606 and the lower housing 604. The locking piece 618 is fixedly attached with a distal end of the drive spindle 606 to prevent any movement of the drive spindle 606 relative to the locking piece 618. Further, the locking piece 618 can transition between a locked position and an unlocked position. For example, a distal surface of the locking piece 618 includes a star ratchet that releasably engages a proximal surface of the lower housing 604. The engagement between the locking piece 618 and the lower housing 604, i.e. the locked position, can be maintained by a biasing member (not shown), which applies a longitudinal distal pressure to locking piece 618, holding it against the lower housing 604, biasing the locking piece 618 towards a locked position. It will be appreciated that the engagement between the distal surface of the locking piece 618 and the proximal surface of the lower housing 604 can include a variety of locking mechanisms, for example, protrusions, detents, recesses, hex-key engagement, or similar “lock and key” engagement, or combinations thereof. Further, the locking piece 618 is slidably engaged with the drive screw 610 such that the drive screw 610 can move along a longitudinal axis relative to the locking piece 618. Accordingly, the locking piece 618 and the drive screw 610 can rotate relative to each other without causing any relative longitudinal movement therebetween.

In use, the intraosseous access system 600 is provided with the drive screw 610 in the retracted position, i.e. with the drive screw head 612 adjacent a proximal end of drive spindle cavity 608. The access assembly 109 is disposed within the lower housing cavity 622 with the drive nut 620 disposed adjacent the locking piece 618. In an embodiment, a distal tip 205 of needle 204 can extend beyond a distal surface 630 of the lower housing 604. In an embodiment, a distal tip 205 of needle 204 is disposed within the lower housing 604. The user can place the distal surface 630 of the driver 601 against the skin surface 70 of the patient. The tip 205 of the needle 204 penetrates the skin surface and contacts a surface of the bone cortex 80.

The user then applies sufficient pressure to the proximal surface 632 of the driver 601 so that the needle tip 205 presses against the bone cortex 80. This causes the drive screw 610 and drive nut 620 to slide, relative to the lower housing 604, and press against the locking piece 618. The locking piece can slide longitudinal and disengage the locking piece 618 from the lower housing 604. The energy source 615, e.g. drive spring, can then rotate the locking piece 618 and drive spindle 606 relative to the upper housing 602. The drive screw head 612 and drive screw 610 can then rotate relative to the driver 601, and engage the housing nut 616 to urge the drive screw 610 and access assembly 109 in a distal direction. This urges the needle 204 through bone cortex 80 so that the needle tip 205 can access the medullary cavity 90.

Advantageously, the pressure activation of the intraosseous access device 600 provides a quick and easy deployment of the access assembly 109. There is no need for any assembly of parts, needles, or the like and deployment involves only a single step process of aligning the needle tip and applying pressure to a proximal end 632. Further, applying pressure as such, stabilizes the device against the patient and provides sufficient counter-resistance to drive the needle 204 through the bone cortex 90. The rotation of the drive screw 612 causes the access assembly 109 to rotate, which also provides a drilling action, together with the distal force, and further facilitates driving the needle 204 through the bone cortex 80. In an embodiment, the coupling between the access assembly 109 and the drive screw 612 can allow for free rotation therebetween to offset and rotational movement and only provide distal force to provide a cleaner puncture through the bone cortex 80. Advantageously, an electric motor energy source 615 can provide a consistent force between the start and finish of the process. Advantageously, a spring driven energy source 615 can be stored for a longer time without loss of power, accordingly, intraosseous access systems 600 would not have to be replaced as often.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An intraosseous system, comprising:
an access assembly, comprising an obturator assembly coupled to a needle assembly; and
an advancement assembly, comprising:
a housing including an inner wall;
a drive screw coupled to an energy source, the drive screw including a threaded portion extending from a drive screw head, the threaded portion extending into the housing;
an advancement nut threadably engaged with the threaded portion of the drive screw, the advancement nut including an outer surface configured to slide along the inner wall a predetermined distance without rotating; and
a socket configured for attachment to the access assembly.

2. The intraosseous system according to claim 1, wherein the access assembly further comprises a needle shield.

3. The intraosseous system according to claim 1, further comprising a driver disposed over the advancement assembly.

4. The intraosseous system according to claim 3, wherein the energy source and the drive screw head are positioned proximal of the housing within the driver.

5. The intraosseous system according to claim 1, wherein the socket is configured for attachment to a proximal end of the obturator assembly.

6. The intraosseous system according to claim 1, wherein the energy source is configured to rotate the drive screw upon activation of the energy source.

7. The intraosseous system according to claim 6, wherein the access assembly is disposed in the housing upon activation of the energy source.

8. The intraosseous system according to claim 6, further comprising a trigger, wherein activating the energy source includes engaging a trigger.

9. The intraosseous system according to claim 1, wherein the predetermined distance is between 1 cm and 3 cm.

10. The intraosseous system according to claim 1, wherein the energy source is one of a drive spring or an electric motor.

11. A method for accessing a medullary cavity, comprising:
attaching an access assembly to an advancement assembly, the advancement assembly comprising:
a housing including an inner wall;
a drive screw coupled to an energy source, the drive screw including a threaded portion extending from a drive screw head, the threaded portion extending into the housing;
an advancement nut threadably engaged with the threaded portion of the drive screw, the advancement nut including an outer surface configured to slide along the inner wall without rotating; and
a socket configured for attachment to the access assembly; and
activating the energy source to rotate the drive screw and advance the advancement nut a predetermined distance along the threaded portion of the drive screw.

12. The method according to claim 11, wherein activating the energy source includes engaging a trigger.

13. The method according to claim 11, wherein activating the energy source includes applying pressure to a distal tip of the access assembly.

14. The method according to claim 11, wherein the energy source is one of a drive spring or an electric motor.

15. The method according to claim 11, wherein the predetermined distance is between 1 cm and 3 cm.
